(19) United States
(12) Patent Application Publication
(10) Pub. No.: US 2021/0135856 A1
(43) Pub. Date: May 6, 2021

(54) BLOCKCHAIN-ENABLED CONTACT CENTER

(71) Applicant: Talkdesk, Inc., San Francisco, CA (US)

(72) Inventor: Jafar Adibi, Los Angeles, CA (US)

(21) Appl. No.: 16/670,279

(22) Filed: Oct. 31, 2019

(51) Int. Cl. H04L 9/08; H04M 3/51; H04L 9/06; G06Q 40/08; G16H 10/60

(52) U.S. Cl. CPC H04L 9/0825; H04M 3/5191; H04L 9/0637; H04L 2209/88; G16H 10/60; H04L 9/0643; H04L 2209/38; G06Q 40/08

(57) ABSTRACT

Disclosed is a contact center that stores data related to a consumer in a blockchain. More specifically, disclosed is a methodology for conducting and concluding a communication with the consumer; creating a record of the communication; encrypting the record of the communication with asymmetric cryptography; and updating the blockchain with the record of the communication.

### BACKGROUND

As well-known and readily-appreciated by skilled artisans, blockchain is a method of storing a list of entries which cannot be changed easily after they are created by using several concepts from cryptography such as digital signatures and hash functions. Blockchain is implemented and managed autonomously utilizing a peer-to-peer network and a distributed timestamping server. This enables the blockchain to be authenticated by the collective collaboration of blockchain users who have shared self-interests in preventing the blockchain from being altered, and thereby provides blockchain users with a high degree of confidence in the security of the data maintained by the blockchain.

Contact centers today are primarily on-premise software solutions. This requires an enterprise to make a substantial investment in hardware, installation and regular maintenance of such solutions. Using on-premise software, agents and supervisors are stationed in an on-site contact center. In addition, a dedicated IT staff is required because on-site software may be too complicated for supervisors and agents to handle on their own. Another drawback of on-premise solutions is that such solutions cannot be easily enhanced to include capabilities to that meet the current demands of technology, such as automation.

An alternative to an on-premise contact center is a cloud-based contact center solution that provides agent automation through the use of machine learning, artificial intelligence, hyperscaling and massive-node parallel processing, and other features cloud computer can efficiently and effectively provide. A cloud-based contact center solution is also further enhanced by the incorporation and utilization of blockchain technology to store contact data and secure the data using asymmetric cryptography for additional security, signature authentication, and so forth.

### SUMMARY

Disclosed herein are various implementations directed to systems, processes, methods, and other implementations for a contact center to store data related to a consumer in a blockchain. More specifically, disclosed herein are various implementations featuring a methodology comprising conducting and concluding a communication with the consumer; creating a record of the communication; encrypting the record of the communication with asymmetric cryptography; and updating the blockchain with the record of the communication.

For example, various implementations disclosed herein are directed to approaches for a contact center to store data related to a consumer in a blockchain comprising: receiving an incoming communication from the consumer; concluding the communication; creating a record of the communication; encrypting the record of the communication with a first key, wherein said record can be decrypted using a second key, the first key and second key operable as an encoder/decoder pair of keys for asymmetric cryptography; and updating the blockchain with the record of the communication.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary and the following detailed description of illustrative implementations are better understood when read in conjunction with the appended drawings. For the purpose of illustrating the implementations, there is shown in the drawings example constructions of the implementations; however, the implementations are not limited to the specific methods and instrumentalities disclosed. In the drawings:

FIG. 1 is a block diagram illustrating an exemplary network-based communications environment pertaining to various implementations described herein;

FIG. 2 is a block diagram illustrating exemplary components that provide automation, routing, and/or omnichannel functionalities within the context of the environment of FIG. 1 pertaining to various implementations described herein;

FIG. 3 is a block diagram illustrating a system for managing a blockchain pertaining to various implementations described herein;

FIG. 4 is a process flow diagram illustrating an exemplary methodology for a contact center to store data in a blockchain corresponding to an incoming communication from a consumer, said methodology representative of various implementations described herein;

FIG. 5 is process flow diagram 500—similar to but distinguishable from the process flow diagram of FIG. 4—illustrating an exemplary methodology for a contact center to store data in a blockchain corresponding to an outgoing communication to a consumer, said methodology representative of various alternative implementations described herein; and

FIG. 6 is a block diagram of an example computing environment that may be used in conjunction with example implementations and aspects herein disclosed.

### DETAILED DESCRIPTION

Certain terms used herein may also be used interchangeably with other terms used herein and such terms should be given the broadest interpretation possible unless explicitly noted otherwise. For example, as used herein, the terms “distributed” and “decentralized” are used interchangeably and the use of either term is intended to fully include the meaning of the other term without limitation.

An understanding of various concepts are helpful to a broader and more complete understanding of the various implementations disclosed herein, and skilled artisans will readily appreciate the implications these various concepts have on the breath and depth of the various implementations herein disclosed.

Contact Centers

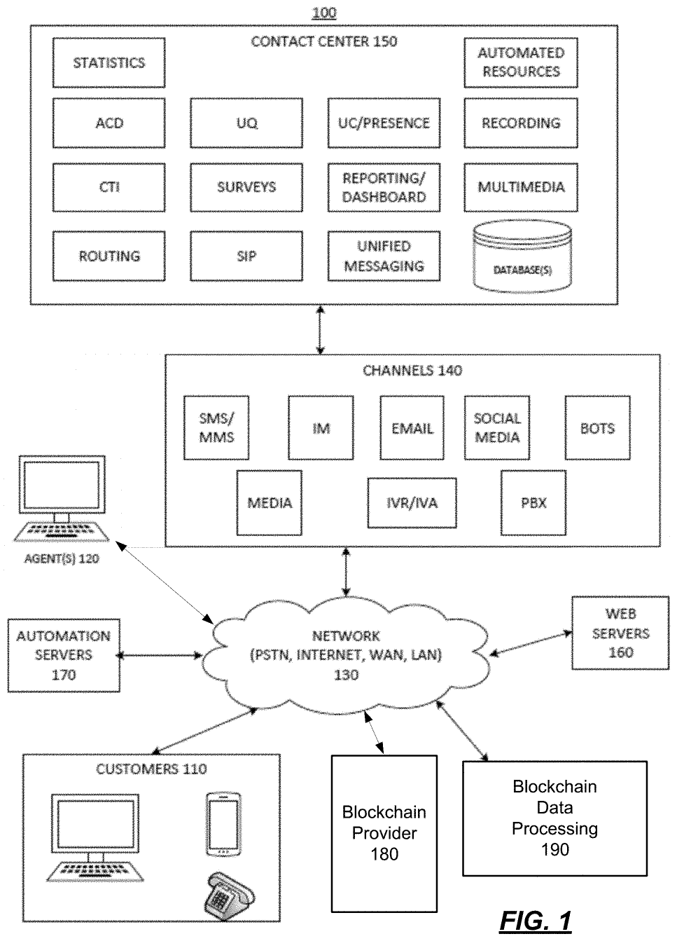

FIG. 1 is an example system architecture 100, and illustrates example components, functional capabilities and optional modules that may be included in a cloud-based contact center infrastructure solution. Customers 110 interact with a contact center 150 using voice, email, text, and web interfaces in order to communicate with agent(s) 120 through a network 130 and one or more of text or multimedia channels 140. The agent(s) 120 may be remote from the contact center 150 and handle communications with customers 110 on behalf of an enterprise. The agent(s) 120 may utilize devices, such as but not limited to, work stations, desktop computers, laptops, telephones, a mobile smartphone and/or a tablet. Similarly, customers 110 may communicate using a plurality of devices, including but not limited to, a telephone, a mobile smartphone, a tablet, a laptop, a desktop computer, or other. For example, telephone communication may traverse networks such as a public switched telephone networks (PSTN), Voice over Internet Protocol (VoIP) telephony (via the Internet), a Wide Area Network (WAN) or a Large Area Network. The network types are provided by way of example and are not intended to limit types of networks used for communications.

The contact center 150 itself be in a single location or may be cloud-based and distributed over a plurality of locations. The contact center 150 may include servers, databases, and other components. In particular, the contact center 150 may include, but is not limited to, a routing server, a SIP server, an outbound server, a reporting/dashboard server, automated call distribution (ACD), a computer telephony integration server (CTI), an email server, an IM server, a social server, a SMS server, and one or more databases for routing, historical information and campaigns.

The reporting server may be configured to generate reports from data aggregated by the statistics server. Such reports may include, but are not limited to, near real-time reports or historical reports concerning the state of resources, such as, average waiting time, abandonment rate, agent occupancy, etc. The reports may be generated automatically or in response to specific requests from a requestor (e.g. agent/administrator, contact center application, etc.).

The routing server may serve as an adapter or interface between the switch and the remainder of the routing, monitoring, and other communication-handling components of the contact center. The routing server may be configured to process PSTN calls, VoIP calls, and the like. For example, the routing server may be configured with the CTI server software for interfacing with the switch/media gateway and contact center equipment. In other examples, the routing server may include the SIP server for processing SIP calls. The routing server may extract data about the customer interaction such as the caller's telephone number (often known as the automatic number identification (ANI) number), or the customer's internet protocol (IP) address, or email address, and communicate with other contact center components in processing the interaction.

The ACD is used by inbound, outbound and blended contact centers to manage the flow of interactions by routing and queuing them to the most appropriate agent. Within the CTI, software connects the ACD to a servicing application (e.g., customer service, CRM, sales, collections, etc.), and looks up or records information about the caller. CTI may display a customer's account information on the agent desktop when an interaction is delivered. Campaign management may be performed by an application to design, schedule, execute and manage outbound campaigns. Campaign management systems are also used to analyze campaign effectiveness.

For inbound SIP messages, the routing server may use statistical data from the statistics server and a routing database to the route SIP request message. A response may be sent to the media server directing it to route the interaction to a target agent 120. The routing database may include: customer relationship management (CRM) data; data pertaining to one or more social networks (including, but not limited to network graphs capturing social relationships within relevant social networks, or media updates made by members of relevant social networks); agent skills data; data extracted from third party data sources including cloud-based data sources such as CRM; or any other data that may be useful in making routing decisions.

Customers 110 may initiate inbound communications (e.g., telephony calls, emails, chats, video chats, social media posts, etc.) to the contact center 150 via an end user device. End user devices may be a communication device, such as, a telephone, wireless phone, smart phone, personal computer, electronic tablet, etc., to name some non-limiting examples. Customers 110 operating the end user devices may initiate, manage, and respond to telephone calls, emails, chats, text messaging, web-browsing sessions, and other multi-media transactions. Agent(s) 120 and customers 110 may communicate with each other and with other services over the network 130. For example, a customer calling on telephone handset may connect through the PSTN and terminate on a private branch exchange (PBX). A video call originating from a tablet may connect through the network 130 terminate on the media server. The channels 140 are coupled to the communications network 130 for receiving and transmitting telephony calls between customers 110 and the contact center 150. A media gateway may include a telephony switch or communication switch for routing within the contact center. The switch may be a hardware switching system or a soft switch implemented via software. For example, the media gateway may communicate with an automatic call distributor (ACD), a private branch exchange (PBX), an IP-based software switch and/or other switch to receive Internet-based interactions and/or telephone network-based interactions from a customer 110 and route those interactions to an agent 120. More detail of these interactions is provided below.

As another example, a customer smartphone may connect via the WAN and terminate on an interactive voice response (IVR)/intelligent virtual agent (IVA) components. IVR are self-service voice tools that automate the handling of incoming and outgoing calls. Advanced IVRs use speech recognition technology to enable customers 110 to interact with them by speaking instead of pushing buttons on their phones. IVR applications may be used to collect data, schedule callbacks and transfer calls to live agents. IVA systems are more advanced and utilize artificial intelligence (Al), machine learning (ML), advanced speech technologies (e.g., natural language understanding (NLU)/natural language processing (NLP)/natural language generation (NLG)) to simulate live and unstructured cognitive conversations for voice, text and digital interactions. IVA systems may cover a variety of media channels in addition to voice, including, but not limited to social media, email, SMS/MMS, IM, etc. and they may communicate with their counterpart's application (not shown) within the contact center 150. The IVA system may be configured with a script for querying customers on their needs. The IVA system may ask an open-ended questions such as, for example, “How can I help you?” and the customer 110 may speak or otherwise enter a reason for contacting the contact center 150. The customer's response may then be used by a routing server to route the call or communication to an appropriate contact center resource.

In response, the routing server may find an appropriate agent 120 or automated resource to which an inbound customer communication is to be routed, for example, based on a routing strategy employed by the routing server, and further based on information about agent availability, skills, and other routing parameters provided, for example, by the statistics server. The routing server may query one or more databases, such as a customer database, which stores information about existing clients, such as contact information, service level agreement requirements, nature of previous customer contacts and actions taken by contact center to resolve any customer issues, etc. The routing server may query the customer information from the customer database via an ANI or any other information collected by the IVA system.

Once an appropriate agent and/or automated resource is identified as being available to handle a communication, a connection may be made between the customer 110 and an agent device of the identified agent 120 and/or the automate resource. Collected information about the customer and/or the customer's historical information may also be provided to the agent device for aiding the agent in better servicing the communication. In this regard, each agent device may include a telephone adapted for regular telephone calls, VoIP calls, etc. The agent device may also include a computer for communicating with one or more servers of the contact center and performing data processing associated with contact center operations, and for interfacing with customers via voice and other multimedia communication mechanisms.

The contact center 150 may also include a multimedia/social media server for engaging in media interactions other than voice interactions with the end user devices and/or other web servers 160. The media interactions may be related, for example, to email, vmail (voice mail through email), chat, video, text-messaging, web, social media, co-browsing, etc. In this regard, the multimedia/social media server may take the form of any IP router conventional in the art with specialized hardware and software for receiving, processing, and forwarding multi-media events.

The web servers 160 may include, for example, social media sites, such as, Facebook, Twitter, Instagram, etc. In this regard, the web servers 160 may be provided by third parties and/or maintained outside of the contact center 160 that communicate with the contact center 150 over the network 130. The web servers 160 may also provide web pages for the enterprise that is being supported by the contact center 150. End users may browse the web pages and get information about the enterprise's products and services. The web pages may also provide a mechanism for contacting the contact center, via, for example, web chat, voice call, email, WebRTC, etc.

The integration of real-time and non-realtime communication services may be performed by unified communications (UC)/presence sever. Real-time communication services include Internet Protocol (IP) telephony, call control, instant messaging (IM)/chat, presence information, real-time video and data sharing. Non-real-time applications include voicemail, email, SMS and fax services. The communications services are delivered over a variety of communications devices, including IP phones, personal computers (PCs), smartphones and tablets. Presence provides real-time status information about the availability of each person in the network, as well as their preferred method of communication (e.g., phone, email, chat and video).

Recording applications may be used to capture and play back audio and screen interactions between customers and agents. Recording systems should capture everything that happens during interactions and what agents do on their desktops. Surveying tools may provide the ability to create and deploy post-interaction customer feedback surveys in voice and digital channels. Typically, the IVR/IVA development environment is leveraged for survey development and deployment rules. Reporting/dashboards are tools used to track and manage the performance of agents, teams, departments, systems and processes within the contact center. Reports are presented in narrative, graphical or tabular formats. Reports can be created on a historical or real-time basis, depending on the data collected by the contact center applications. Dashboards typically include widgets, gadgets, gauges, meters, switches, charts and graphs that allow role-based monitoring of agent, queue and contact center performance. Unified messaging (UM) applications include various messaging and communications media (voicemail, email, SMS, fax, video, etc.) stored in a common repository and accessed by users via multiple devices through a single unified interface.

The cloud-based contact center 150 may include a number of other components. For example, the cloud-based contact center 150 may provide for speech analytics (e.g., post-call and real-time) to capture, structure and analyze unstructured phone conversations to uncover the reasons why people call, and to allow a company to identify and address an issue while the caller is still on the line. Text analytics may be used to extract information from unstructured text-based data such as emails, chats, SMS, social media, etc., in order to structure it for further analysis or action. Robotic process automation (RPA) may leverage artificial intelligent (Al), machine learning, workflow and other technologies to automate the processing of repetitive tasks, initiate actions and communicate with other systems or employees. RPA emulates the processes performed by human workers and can be trained to adapt to changing conditions, anomalies and new situations. Desktop analytics (DA) may capture, track and analyze events on the agent desktop. Real-time guidance/next-best action (NBA) tools may give agents the right information at the right time to deliver a personalized experience to each customer.

Automation operations may be used to enhance the operation of the contact center 150. In one aspect, the automation operations may be implemented as an application running on a mobile device of a customer 110, one or more cloud computing devices (generally labeled automation servers 170 connected to the end user device over the network 130), one or more servers running in the contact center 150 (e.g., automated resources), or combinations thereof.

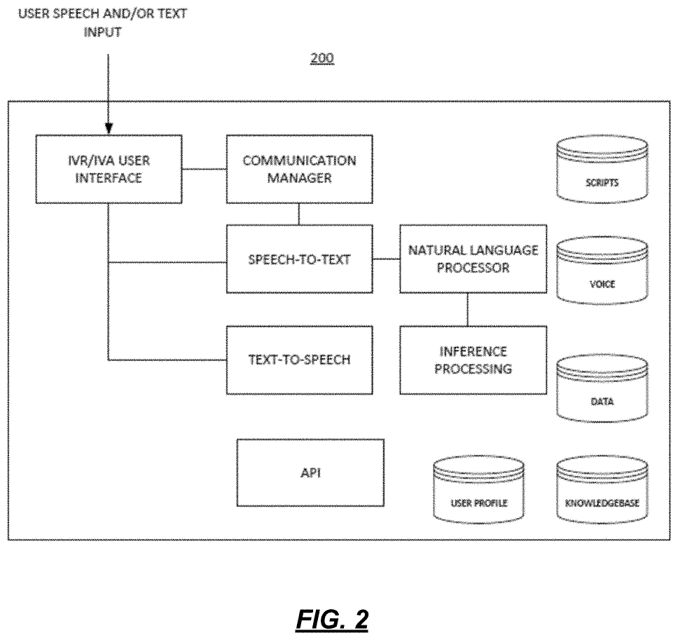

FIG. 2 illustrates an example automation application infrastructure 200 that may be implemented as the one or more automation servers 170 and/or the server(s) within the cloud-based contact center 150. The automation infrastructure 200 may automatically collect information from a customer 110 user through, e.g., a user interface, where the collection of information does not require the involvement of a live agent. The user input may be provided as free speech or text (e.g., unstructured, natural language input). This information may be used by the application 200 for routing the customer 110 to an agent 120, to automated resources in the contact center 150, as well as gathering information from other sources to be provided to the agent 120. In operation, the application 200 may parse the natural language user input using a natural language processing module from the system 200 to infer the customer's intent using an intent inference module in order to classify the intent. Where the user input is provided as speech, the speech is transcribed into text by a speech-to-text system (e.g., a large vocabulary continuous speech recognition or LVCSR system) as part of the parsing by the natural language processing module.

The intent inference module of the application 200 automatically infers the customer's 110 intent from the text of the user input using artificial intelligence or machine learning techniques. These artificial intelligence techniques may include, for example, identifying one or more keywords from the user input and searching a database of potential intents (e.g., call reasons) corresponding to the given keywords. The database of potential intents and the keywords corresponding to the intents may be automatically mined from a collection of historical interaction recordings, in which a customer may provide a statement of the issue, and in which the intent is explicitly encoded by an agent.

Various implementations disclosed herein may feature automatically navigating an IVR system of a contact center on behalf of a user using, for example, the loaded script. In some implementations of the present disclosure, the script includes a set of fields (or parameters) of data that are expected to be required by the contact center in order to resolve the issue specified by the customer's 110 intent. In some implementations, some of the fields of data are automatically loaded from a stored user profile. These stored fields may include, for example, the customer's 110 full name, address, customer account numbers, authentication information (e.g., answers to security questions) and the like.

Various implementations disclosed herein may feature the automatic authentication of the customer 110 with the provider. For example, in some implementations of the present disclosure, the user profile may include authentication information that would typically be requested of users accessing customer support systems such as usernames, account identifying information, personal identification information (e.g., a social security number), and/or answers to security questions. As additional examples, the application 200 may have access to text messages and/or email messages sent to the customer's 110 account on the end user device in order to access one-time passwords sent to the customer 110, and/or may have access to a one-time password (OTP) generator stored locally on the end user device. Accordingly, implementations of the present disclosure may be capable of automatically authenticating the customer 110 with the contact center prior to an interaction.

In some implementations of the present disclosure an application programming interface (API) is used to interact with the provider directly. The provider may define a protocol for making commonplace requests to their systems. This API may be implemented over a variety of standard protocols such as Simple Object Access Protocol (SOAP) using Extensible Markup Language (XML), a Representational State Transfer (REST) API with messages formatted using XML or JavaScript Object Notation (JSON), and the like. Accordingly, a customer experience automation system 200 according to one implementation of the present disclosure automatically generates a formatted message in accordance with an API define by the provider, where the message contains the information specified by the script in appropriate portions of the formatted message.

Various implementations disclosed herein may feature systems and methods for automating and augmenting aspects of an interaction between the customer 110 and a live agent of the contact center. In an implementation, once an interaction, such as through a phone call, has been initiated with the agent 120, metadata regarding the conversation is displayed to the customer 110 and/or agent 120 in the UI throughout the interaction. Information, such as call metadata, may be presented to the customer 110 through the UI 205 on the customer's 110 mobile device 105. Examples of such information might include, but not be limited to, the provider, department call reason, agent name, and a photo of the agent.

According to some aspects of implementations of the present disclosure, both the customer 110 and the agent 120 can share relevant content with each other through the application (e.g., the application running on the end user device). The agent may share their screen with the customer 110 or push relevant material to the customer 110.

In yet another implementation, the application 200 may also “listen” in on the conversation and automatically push relevant content from a knowledge base to the customer 110 and/or agent 120. For example, the application may use a real-time transcription of the customer's input (e.g., speech) to query a knowledgebase to provide a solution to the agent 120. The agent may share a document describing the solution with the customer 110. The application may include several layers of intelligence where it gathers customer intelligence to learn everything it can about why the customer 110 is calling. Next, it may perform conversation intelligence, which is extracting more context about the customer's intent. Next, it may perform interaction intelligence to pull information from other sources about customer 100. The application 200 may also perform contact center intelligence to implement WFM/WFO features of the contact center 150.

Blockchain Technologies

Blockchain is a method of storing a list of entries which cannot be changed easily after they are created by using several concepts from cryptography such as digital signatures and hash functions. To create a blockchain block, a checksum is calculated over a defined block of data using special hash functions that are designed to return a value of a defined length (the hash value or “message digest”) which is not dependent on the length of the input (i.e., the defined block of data) but which does provide the same output when provided with the same input. In addition to the hash values, the blockchain block will also typically include a timestamp, a payload (i.e., the defined data block), and a digital signature for detecting any changes made to the data after to the digital signature is created. When added to the blockchain, the newly created blockchain block will also contain the hash value of the immediately previous block in the blockchain, thereby creating an up-chain link to that previous block.

Blockchain is generally implemented on and managed by a peer-to-peer network where all peers use a common protocol that specifies how each peer should communicate with the other peers and how a new block is created and validated. More specifically, blockchain is typically implemented as a decentralized, distributed, and public digital ledger used to record transactions across many computers where the records associated with the blockchain cannot be surreptitiously altered retroactively (that is, after the records are committed) without also surreptitiously altering all of the subsequent blocks in the chain—a nearly impossible task. Blockchain also allows user to verify and audit transactions independently and relatively inexpensively. In this manner, data stored on the blockchain is generally considered incorruptible. In the context of digital currency, for example, blockchain can also be used to prevent infinite reproducibility of a digital asset by confirming that each unit of value was transferred only once by maintaining a record of offer and acceptance resulting in the transaction.

In a blockchain system, each peer-to-peer computer system comprises a node in the decentralized system, each having a copy of the entire blockchain and its history. Data quality is maintained by massive database replication and computational trust where no centralized “official” copy exists and no user is “trusted” more than any other. Transactions are broadcast to the network, and messages are delivered on a best-efforts basis. Completed blocks are broadcast to other nodes, and various time-stamping schemes are used to serialize changes to the blockchain. Over time, however, computer resources required to process the larger and larger amounts of data in the blockchain become less efficient and more expensive.

In blockchain, blocks hold batches of valid transactions that are hashed and encoded into a defined structure such as a Merkle tree. Each block in the blockchain includes the cryptographic hash of the prior block in the blockchain, logically linking both blocks to form part of a larger chain of blocks, wherein each block confirms the integrity of the previous block all the way up the chain to the original block (a.k.a., the “genesis block”). In this manner, a blockchain maintains a secure hash-based history.

By adding new blocks to the blockchain—and given the blockchain's distributed nature—a temporary fork in the blockchain may result when separate blocks are produced concurrently, resulting in different versions of the blockchain history as to each of the concurrently produced blocks. To reconcile this situation, a blockchain uses a special algorithm (e.g., a proof-of-work algorithm) for scoring the different versions of the blockchain history for each block in the fork and then selects the block (and associated blockchain history) having the highest score. The blocks not selected for inclusion in the chain become orphan blocks. Notably, orphan blocks are still valid blocks that meet all of the requirements for addition to the blockchain but are simply rejected to resolve the temporary fork. Thus, while the selected block—that is, the one that had the highest score—becomes the block upon which subsequent building of the block chain will be continued, orphan blocks are become stale (unused and not included) and the contents of such blocks must be resubmitted in new blocks for inclusion further down the blockchain. In other words, any transactions present in an orphan block (and not included in the selected block) will be included in a subsequent block for subsequent addition to the blockchain.

Similarly, peer-to-peer computing systems supporting the blockchain database (each a “peer”) may have different versions of the blockchain history from time to time with each peer keeping only the highest-scoring version of the database known to it at any given time. Then when a peer receives a higher-scoring version of the blockchain history—oftentimes the previous version of the blockchain but with a single new block having been added—the peer simply extends (or overwrite) its own database and then passes along the updated database to other peers.

By distributing data across a peer-to-peer network, blockchain eliminates a number of risks that would otherwise stem from the data being held centrally. For example, peer-to-peer blockchain networks lack centralized points of failure and centralized points of vulnerability that hackers might otherwise exploit. Blockchain may also utilize additional security methods such public-key cryptography where a public key, appearing as a long string of seemingly-random numbers, is in fact an address for a block on the blockchain, and where a private key acts as a password giving its owner access to the digital assets of that particular block or the means to otherwise interact with the various capabilities that blockchains now support with regard to that particular block (e.g., value tokens being recorded as belonging to the block having said address).

Blockchains are secure by design, and thus blockchain technology can be useful in a variety of different endeavors. Presently the primary use of blockchains is as a distributed ledger for cryptocurrencies, and most cryptocurrencies today use blockchain technology to record transactions. Similarly, blockchain can also be used for “smart contracts” that can be partially or fully executed and/or enforced without human interaction, with one of the main objectives for smart contract being an automated escrow. In addition, the financial services industry is interested in implementing distributed ledgers for use in banking because of the potential for blockchain technologies to speed up back office settlement systems. There are also efforts to employ blockchains in supply chain logistics and management, and online voting is another potential application for blockchain. Blockchain can also be used to secure medical records, identity management applications, and support traceability of food, drugs, and other safety-monitored consumables.

In blockchain, cryptography is primarily used for two purposes: to secure the identity of the sender of a transaction and to ensure the past records have not been altered.

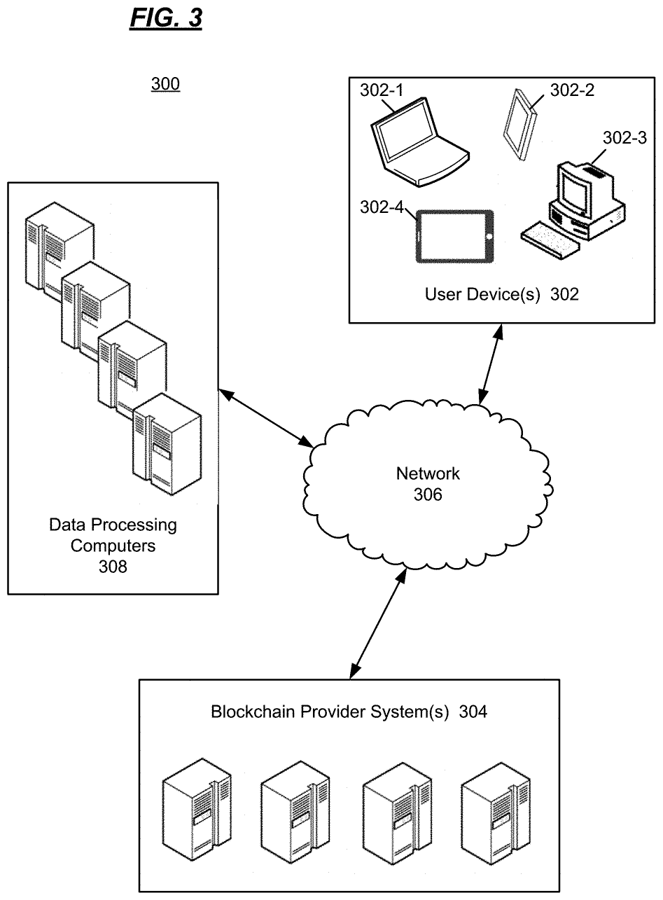

FIG. 3 is a block diagram illustrating a system for managing a blockchain pertaining to various implementations described herein. The system 300 may comprise user device(s) 302 (e.g., user device 302-1, user device 302-2, user device 302-3, and user device 302-4). It should be appreciated that user device(s) 302 may comprise any suitable number of user devices. The system 300 may further include a blockchain provider system(s) 304 and data processing computer(s) 308. Each of these systems and computers may be communicatively coupled with each other (e.g., via the network 306). For simplicity of illustration, a certain number of components are shown in FIG. 3. It is understood, however, that embodiments of the invention may include more than one of each component. In addition, some embodiments of the invention may include fewer than or greater than all of the components shown in FIG. 3. In addition, the components in FIG. 3 may communicate via any suitable communication medium (including the Internet), using any suitable communications protocol.

The blockchain provider system(s) 304 may have any suitable characteristics. The blockchain provider system(s) 304 may individually include a processor and a computer readable medium coupled to the processor, the computer readable medium comprising code, executable by the processor for performing the functionality described herein. The blockchain provider system(s) 304 may be communicatively coupled to the data processing computer(s) 308 (e.g., via network 306). In at least some embodiments, the blockchain provider system(s) 304 may additionally be communicatively coupled to the user device(s) 302 (e.g., via the network 306).

In at least one embodiment, the blockchain provider system(s) 304 may be configured to perform token management functions including the maintenance and/or enforcement of tokens (e.g., an amount and/or threshold limit associated with a user and/or channel). In at least one example, the blockchain provider system(s) 304 may be configured to receive token request messages from the data processing computer(s) 308 and/or the user device(s) 302 and provide token response messages to the data processing computer(s) 308 and/or the user device(s) 302. In some embodiments, the blockchain provider system(s) 304 may be configured to provision and maintain a mapping of a token (e.g., an amount and/or threshold limit) and a user/entity for which the token is associated.

In at least one embodiment, the blockchain provider system(s) 304 may further be configured to receive data transfer request message(s) from the user device(s) 302, individually, and/or from the data processing computer(s) 308 (e.g., via the network 306). The blockchain provider system(s) 304 may further be configured to perform functions including managing one or more ledgers according to received data transfer request messages and/or token request messages.

The user device(s) 302 may individually include a processor and a computer readable medium coupled to the processor, the computer readable medium comprising code, executable by the processor for performing the functionality described herein. The user device(s) 302 may be communicatively coupled to the data processing computer(s) 308 and/or the blockchain provider system(s) 304 via a communications medium (e.g., the network 306) in order to exchange information (e.g., interaction records). The user device(s) 302 may individually include one or more software modules that comprise a software application (e.g., a data transfer application). The software application may be configured to manage information related to data transfers initiated and/or received by the user device(s) 302.

The data processing computer(s) 308 may be associated with any suitable data processing provider. The data processing computer(s) 308 may individually include a processor and a computer readable medium coupled to the processor, the computer readable medium comprising code, executable by the processor for performing the functionality described herein. Examples of data processing computer(s) 308 includes any device capable of communicating with the network 306 and initiating/processing information, including transfer channel request/response messages, token request/response messages, and/or data transfer request/response messages. The data processing computer(s) 308 may transmit and/or receive data through the communications medium (e.g., the network 306) to/from at least one of user device(s) 302 and/or the blockchain provider system(s) 304. In at least one embodiment, the data processing computer(s) 308 may be configured to manage an electronic record associated with one or more users/user device(s) 302. The electronic record, in some cases, may include one or more interaction records associated with a data request/response message. In still further examples, the data processing computer(s) 308 may be configured to enforce one or more threshold limits associated with one or more users/user device(s) 302. In at least one embodiment, the data processing computer(s) 308 may be configured to generate and store public and/or private keys for at least one of the user device(s) 302. The data processing computer(s) 308 may utilize the stored public key associated with a user device to verify messages (e.g., a transfer channel request/response message, a data transfer request/response message) from the user device in order to validate a message initiated by the user device.

In at least one embodiment, the data processing computer(s) 308 may be configured to facilitate the exchange of transfer channel request/response messages and/or data transfer request/response messages between two or more of the user device(s) 302. For example, the data processing computer(s) 308 may be configured to act as an intermediary device to facilitate the exchange of transfer channel request/response messages and/or data transfer request/response messages via transfer channels A, B, C, and D as depicted in FIG. 3. In one non-limiting example, the user device 302-1 may submit a transfer channel request message (e.g., via an application operating on the user device 302-1) that requests a transfer channel be created. Upon receipt, or at another suitable time, the data processing computer(s) 308 may forward the transfer channel request message to the user device 302-3. A user of the user device 302-3 may indicate (e.g., utilizing an application operating on the user device 302-3) whether or not he wishes to allow the creation of a transfer channel (e.g., an electronic record) associated with the user device 302-1 and the user device 302-3. If the user of the user device 302-3 rejects the transfer channel request, the data processing computer(s) 308 may be configured to refrain from further processing. However, if the user of the user device 302-3 approves the transfer channel request, a transfer channel response message may be transmitted from the user device 302-3 to the data processing computer(s) 308. In at least one embodiment, the transfer channel request messages may be exchanged directly between the user device 302-1 and the user device 302-3. In these examples, the transfer channel response message may be transmitted by one, or both user devices, and received by the data processing computer(s) 308.

In at least one embodiment, upon receipt of a transfer channel response message that indicates that a transfer channel has been initiated and approved, the data processing computer(s) 308 may be configured to create an electronic record associated with the user device 302-1 and the user device 302-3. The process of creating the electronic record associated with the user device 302-1 and the user device 302-3 may be referred to as “opening a channel.” In some examples, the data processing computer(s) 308 may additionally receive, via the transfer channel request message and/or the transfer channel response message, one or more threshold limits. Each threshold limit may be associated with the user device 302-1, the user device 302-3, or the electronic record.

In some examples, the data processing computer(s) 308 may be configured to request one or more tokens from the blockchain provider system(s) 304 on behalf of the user device 302-1 and/or the user device 302-3. A token may be associated with an amount and/or threshold limit received from a transfer channel request message. In some examples, the data processing computer(s) 308 may wait until a token response message (e.g., indicating that the amount and/or threshold limit of the token has been recorded) is received for each of the devices associated with the transfer channel before opening a transfer channel (e.g., creating an electronic record associated with the user devices). In at least one embodiment, the received token response messages may individually include a token (e.g., and amount and/or a threshold limit) for which a user's data transfer amounts cannot exceed. In other examples, the received token(s) may individually represent a threshold limit for which a combined data transfer amount associated with a set of data transfers recorded within the electronic record cannot exceed.

After creating the electronic record associated with the user device 302-1 and the user device 302-3, the data processing computer(s) 308 may be configured to receive data transfer request messages from either/both the user device 302-1 and/or the user device 302-3. Upon receipt of each data transfer request message, the data processing computer(s) 308 may be configured to forward the data transfer request message to the other user device. Upon receiving a data transfer response message from the other device indicating that the data transfer request was approved, the data processing computer(s) 308 may be configured to record the data transfer as an interaction record within the electronic record associated with the user devices.

In at least one embodiment, at least one of the user device 302-1 or the user device 302-3 may initiate a transfer channel request message that indicates a desire to close the transfer channel associated with the user device 302-1 and the user device 302-3. In some cases, the data processing computer(s) 308 may be configured to maintain the transfer channel (e.g., the electronic record) until a transfer channel request message has been received from each recipient of data. By way of example, consider that a transfer channel was opened between the user device 302-1 and the user device 302-3, but only the user device 302-1 received any data corresponding one or more data transfer request messages initiated by the user device 302-3. In this example, the transfer channel may be closed (e.g., the electronic record may be deleted) upon receipt of a transfer channel request message from the user device 302-1 requesting that the transfer channel be closed. If both devices were to receive data from the other device, then in some examples, a transfer channel request message requesting that the transfer channel be closed would be required from both user devices before the transfer channel would actually be closed (e.g., the electronic record deleted).

In at least one example, the data processing computer(s) 308, upon closing a channel, or at another suitable time, may be configured to determine a net transfer amount indicating a one-way transfer of data between the user device 302-1 and the user device 302-3. For example, in some cases the user devices may be exchanging cybercurrency and/or fiat currency. Upon closing the channel, the data processing computer(s) 308 may be configured to determine a net transfer amount to be exchanged. The data processing computer(s) 308, in some embodiments, may provide the net transfer amount to the blockchain provider system(s) 304 in a single message (e.g., a data transfer request message). In some examples, the data processing computer(s) 308 may be configured to provide the net transfer amount via the data transfer request message in a format suitable for immediate recordation in the ledger. Upon receipt of the data transfer request message, the blockchain provider system(s) 304 may be configured to record the net transfer amount in a ledger suitable for recording such information. In some cases, the blockchain provider system(s) 304 may be configured to transmit a data transfer response message to the data processing computer(s) 308 to indicate whether recording the net transaction amount within the ledger was successful or unsuccessful.

It should be appreciated that any reference to “opening a channel” may, in some embodiments include creation of an electronic record that may be associated with two or more channel participants. While a channel is open (the electronic record exists), any suitable number of transactions (e.g., information included in a data transfer request/response message) may be recorded in the electronic record. Any reference to “closing a channel” may, in some embodiments include deletion of a previously-created electronic record that may be associated with two or more channel participants. Prior to closing a channel (e.g., deleting the electronic record), amounts corresponding to any suitable number of transactions recorded in the electronic record may be aggregated and information related to the aggregation (e.g., an overall transaction amount) may be provided (e.g., to a blockchain provider system).

In at least one embodiment, messages between the computers, networks, and devices in FIG. 3 may be transmitted using a secure communications protocols such as, but not limited to, File Transfer Protocol (FTP); HyperText Transfer Protocol (HTTP); Secure Hypertext Transfer Protocol (HTTPS), Secure Socket Layer (SSL), ISO (e.g., ISO 8583) and/or the like.

Each of the entities in FIG. 3 may communicate through any suitable communication channel or communications network. A suitable communications network (e.g., the network 306) may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like.

Blockchain Encryption and Digital Signatures

Blockchain makes extensive use of cryptography to ensure transactions are done securely while also securing all information and value stores. In most instances, asymmetric “public-key” cryptography is considerably better suited to the functions associated with blockchain than symmetric key cryptography, the primary difference being that asymmetric cryptography allows information to be encoded and decoded through a pair of keys (e.g., one public key and one private key) where only one key needs to be shared with another party for secure communications in a single direction (and a separate single pair of keys for communications in the other direction), whereas symmetric key cryptography uses only a single key for communications in both direction that is less robust and cannot be so easily shared. Accordingly, rather than using a single key for encryption and decryption—as is the case with symmetric key cryptography—separate keys (generally termed a public key and a private key) are used.

For asymmetric cryptography, the sender uses a combination of the recipient's public key and the sender's own private key to encrypt the information it wants to securely transmit, and the recipient uses its own private key and the sender's public key to decrypt the information. It is practically impossible to determine the private key corresponding to the public key, and so a user can send their public key to anyone without worrying that someone will gain access to their private key. As such, the sender can encrypt files that they can be sure will only be decrypted by the intended receiving party.

Furthermore, through asymmetric cryptography a digital signature can be produced, securing the integrity and validity of the data that is being transmitted. This is done by combining a sender's private key with the data that they wish to sign which can only be decrypted using the sender's public key, and thus the recipient of the digitally-signed transmission can be assured that the transmission is indeed from the sender.

Moreover, because the payload data itself is part of the digital signature—i.e., a part of the decryption necessary for proof of integrity and validity—tampered with any part of the encrypted message will render it invalid because editing even the most aspect of the encrypted data reshapes the whole of the encrypted data including the signature.

Digital signatures are one of the main aspects of ensuring the security and integrity of the data that is recorded onto a blockchain. They are a standard part a blockchain protocols, mainly used for securing transactions and blocks of transactions, transferral of information, contract management, and any other cases where detecting and preventing any external tampering is important. Through use of digital signatures—blockchain is able to guarantee that any data recorded to the blockchain is valid, accurate, and untampered based on the asymmetric cryptography that is maintained from one block to another, the digital signature aspect thereof giving the data recorded on a blockchain its immutability.

Digital signatures provide three key advantages of storing and transferring information on a blockchain. First, digital signatures guaranteed integrity because data cannot be altered without also altering the digital signature. Second, digitally signed encrypted data is not only secure from discovery but will also reveal if it has been tampered with (affirming its incorruptibility) even when the security of the original data itself has not been compromised. Additionally, digital signatures can also affirm the identity of the individual or entity sending the data because a private key digital signature cannot be faked because of the ability to validate the signature with the corresponding public key.

Additionally, because private keys are linked to individual users, digital signatures using private keys cannot be later repudiated. This means that if something is digitally signed by a user, it can be legally binding on that individual, although this is entirely dependent on the private key that signed the data not being otherwise compromised.

Blockchain-Enabled Contact Center

Disclosed herein are various implementations directed to systems, processes, methods, and other implementations for a contact center to store data related to a consumer in a blockchain.

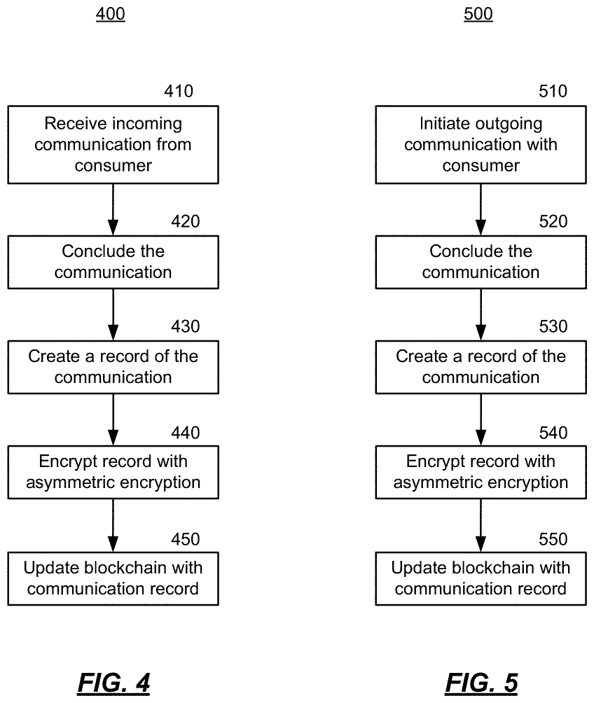

FIG. 4 is a process flow diagram 400 illustrating an exemplary methodology for a contact center to store data in a blockchain corresponding to an incoming communication from a consumer, said methodology representative of various implementations described herein. In FIG. 4, at 410 a contact center receives an incoming communication from a consumer, and at 420 the contact center concludes the communication in order to create a record of the communication at 430, encrypt the record using asymmetric cryptography at 440, and update the blockchain with the record of communication at block 450.

FIG. 5 is process flow diagram 500—similar to but distinguishable from the process flow diagram 400 of FIG. 4—illustrating an exemplary methodology for a contact center to store data in a blockchain corresponding to an outgoing communication to a consumer, said methodology representative of various alternative implementations described herein. In FIG. 5, at 510 a contact center initiates an outgoing communication with a consumer, and at 520 the contact center concludes the communication in order to create a record of the communication at 530, encrypt the record using asymmetric cryptography at 540, and update the blockchain with the record of communication at block 550.

With regard to FIG. 4 and FIG. 5, various implementations disclosed herein may manage the asymmetric cryptography in several different manners depending on the intended objective. For example, the first key may be a public key for the contact center and the second key may be a private key for the contact center such that the record is available on the blockchain but can only be understood (that is, decrypted) by the contact center despite the encrypted data being widely accessible in its encrypted form. Conversely, the first key may be a public key for the consumer and the second key may be a private key for the consumer such that the record is available on the blockchain but can only be accessed (that is, decrypted) by the consumer. Furthermore, the first key may instead be a public key shared by both the contact center and the consumer and the second key may be a private key shared by both the contact center and the consumer such that the record is available on the blockchain but can be accessed (that is, decrypted) by the contact center and the consumer but no one else.

In addition, various implementations disclosed herein may instead (or also) implement the asymmetric cryptography to provide a digital signature. For example, the first key may be a private key for the contact center and the second key may be a public key for the contact center such that the record is thereby digitally signed by the contact center and can later be verified by the contact center's public key. Conversely, the first key may be a private key for the consumer that the consumer has shared with the contact center (in order to create the electronic signature as to the consumer) and the second key may be a public key for the consumer such that the record is thereby digitally signed by the consumer (although created by the contact center on the consumer's behalf) and the consumer's signature can later be verified by the consumer's public key. Similarly, the first key may be a private key shared by contact center and the consumer and the second key may be a public key also shared by the contact center and the consumer such that the record is thereby digitally signed by both the contact center and the consumer where the shared signature can later be verified by the shared public key.

Alternatively, asymmetric encryption can also be utilized where neither key is made public but instead the keys are both private and merely shared by the contact center and/or the consumer in varying arrangements suited to different purposes as between these parties. For example, the first key (a private key) may be associated with the contact center (e.g., private to the contact center and not known to the consumer) and the second key (also a private key) may also be associated with the contact center (private to the contact center and not known to the consumer) in order for the contract center to maintain exclusive control over the secured content which it can then share with other entities (including the consumer if it chooses) by providing these other entities the second key (thereby making it a shared decryption key as opposed to a public decryption key)—an approach that my be beneficial in sharing records that are sensitive in nature or that warrant enhanced security (such as medical records and other HIPPA-related data). Conversely, the first key (the encryption key) may be associated with the contract center (and ostensibly not known to the consumer) while the second key may be associated only with the consumer (and not known to the contact center) in order for the consumer to maintain exclusive control over the secured content which it can then share with other entities (including the contact center if it chooses) by providing these other entities with the second key (thereby again making it a shared decryption key as opposed to a public decryption key)—an approach that my be beneficial in empowering the consumer in sharing records that are sensitive in nature or that warrant enhanced security (such as medical records and other HIPPA-related data) while also centralizing those records with the consumer (instead of the contact center). Furthermore, the first key may be a private key associated with the contact center and the second key may be a private key associated with both the contact center and the consumer such that the record is available on the blockchain but can be accessed (that is, decrypted) by the contact center and the consumer and further shared by either the contact center or the consumer by sharing the second key.

In addition, various implementations disclosed herein may instead (or also) implement the asymmetric cryptography to provide a shared digital signature—that is, a digital signature shared by both the contact center and the consumer based on the first key, and the second key associated with the consumer, the contact center, or both for utilization and sharing with third party entities to (non-publically) validate the digital signature. More specifically, the first key may be associated with the consumer and the contact center and the second key is associated with the consumer (where the consumer, not the contact center, has control over the decryption key); or the first key may be associated with the consumer and the contact center and the second key is associated with the contact center (where the contract center, not the consumer, has control over the decryption key); or the first key may be associated with the consumer and the contact center and the second key is associated with the consumer and the contact center (where both the consumer and the contact center have control over the decryption key and can each independently share that second key with third parties and/or utilize it themselves).

Furthermore, several implementations disclosed herein may be specifically directed to the contact center and consumer in any of several medical context—where the updated blockchain might need to be HIPPA-compliant—such as, for example, with the consumer being a patient and the contact center being associated with a medical service provider, medical insurance provider, a pharmacy, and so forth; or with the consumer being a medical service provider and the contact center being associated with a medical insurance provider or a pharmacy; or with the consumer being a medical insurance provider and the contact center being associated with a pharmacy; or other such combinations and permutations of various entities in the medical field known and appreciated by skilled artisans.

Computing Environment

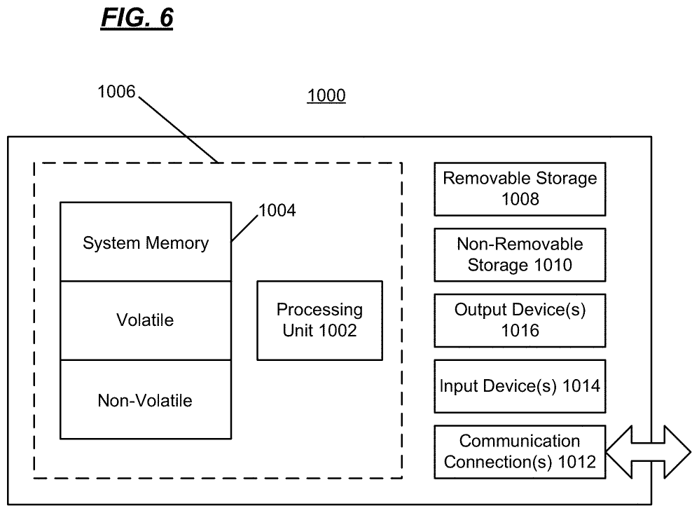

FIG. 6 is a block diagram of an example computing environment 1000 that may be used in conjunction with the various implementations disclosed herein. Computing environment 1000 is only one example of a suitable computing environment and is not intended to suggest any limitation as to the scope of use or functionality.

Numerous other general purpose or special purpose computing system environments or configurations may be used. Examples of well-known computing systems, environments, and/or configurations that may be suitable for use include, but are not limited to, personal computers (PCs), server computers, handheld or laptop devices, multiprocessor systems, microprocessor-based systems, network PCs, minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, and so forth that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 6, an exemplary system for implementing aspects described herein includes a computing device, such as computing device 1000. In a basic configuration, computing device 1000 typically includes at least one processing unit 1002 and memory 1004. Depending on the exact configuration and type of computing device, memory 1004 may be volatile (such as random access memory (RAM)), non-volatile (such as read-only memory (ROM), flash memory, etc.), or some combination of the two. This basic configuration is illustrated in FIG. 6 by dashed line 1006 as may be referred to collectively as the “compute” component.

Computing device 1000 may have additional features/functionality. For example, computing device 1000 may include additional storage (removable and/or non-removable) including, but not limited to, magnetic or optical disks or tape. Such additional storage is illustrated in FIG. 6 by removable storage 1008 and non-removable storage 1010.

Computing device 1000 typically includes a variety of computer readable media. Computer readable media can be any available media that can be accessed by device 1000 and include both volatile and non-volatile media, as well as both removable and non-removable media.

Computer storage media include volatile and non-volatile media, as well as removable and non-removable media, implemented in any method or technology for storage of information such as computer readable instructions, data structures, program modules or other data. Memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the information and which can be accessed by computing device 1000. Any such computer storage media may be part of computing device 1000.

Computing device 1000 may contain communication connection(s) 1012 that allow the device to communicate with other devices. Computing device 1000 may also have input device(s) 1014 such as a keyboard, mouse, pen, voice input device, touch input device, and so forth. Output device(s) 1016 such as a display, speakers, printer, and so forth may also be included. All these devices are well-known in the art and need not be discussed at length here.

Computing device 1000 may be one of a plurality of computing devices 1000 inter-connected by a network. As may be appreciated, the network may be any appropriate network, each computing device 1000 may be connected thereto by way of communication connection(s) 1012 in any appropriate manner, and each computing device 1000 may communicate with one or more of the other computing devices 1000 in the network in any appropriate manner. For example, the network may be a wired or wireless network within an organization or home or the like, and may include a direct or indirect coupling to an external network such as the Internet or the like.

It should be understood that the various techniques described herein may be implemented in connection with hardware or software or, where appropriate, with a combination of both. Thus, the processes and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an API, reusable controls, or the like. Such programs may be implemented in a high level procedural or object-oriented programming language to communicate with a computer system. However, the program(s) can be implemented in assembly or machine language. In any case, the language may be a compiled or interpreted language and it may be combined with hardware implementations.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be affected across a plurality of devices. Such devices might include PCs, network servers, and handheld devices, for example.

Certain implementations described herein may utilize a cloud operating environment that supports delivering computing, processing, storage, data management, applications, and other functionality as an abstract service rather than as a standalone product of computer hardware, software, etc. Services may be provided by virtual servers that may be implemented as one or more processes on one or more computing devices. In some implementations, processes may migrate between servers without disrupting the cloud service. In the cloud, shared resources (e.g., computing, storage) may be provided to computers including servers, clients, and mobile devices over a network. Different networks (e.g., Ethernet, Wi-Fi, 802.x, cellular) may be used to access cloud services. Users interacting with the cloud may not need to know the particulars (e.g., location, name, server, database, etc.) of a device that is actually providing the service (e.g., computing, storage). Users may access cloud services via, for example, a web browser, a thin client, a mobile application, or in other ways. To the extent any physical components of hardware and software are herein described, equivalent functionality provided via a cloud operating environment is also anticipated and disclosed.

Additionally, a controller service may reside in the cloud and may rely on a server or service to perform processing and may rely on a data store or database to store data. While a single server, a single service, a single data store, and a single database may be utilized, multiple instances of servers, services, data stores, and databases may instead reside in the cloud and may, therefore, be used by the controller service. Likewise, various devices may access the controller service in the cloud, and such devices may include (but are not limited to) a computer, a tablet, a laptop computer, a desktop monitor, a television, a personal digital assistant, and a mobile device (e.g., cellular phone, satellite phone, etc.). It is possible that different users at different locations using different devices may access the controller service through different networks or interfaces. In one example, the controller service may be accessed by a mobile device. In another example, portions of controller service may reside on a mobile device. Regardless, controller service may perform actions including, for example, presenting content on a secondary display, presenting an application (e.g., browser) on a secondary display, presenting a cursor on a secondary display, presenting controls on a secondary display, and/or generating a control event in response to an interaction on the mobile device or other service. In specific implementations, the controller service may perform portions of methods described herein.

Additional Disclosures

As used herein, a user device may comprise any suitable electronic device that may be transported and operated by a user, which may also provide remote communication capabilities to a network. Examples of remote communication capabilities include using a mobile phone (wireless) network, wireless data network (e.g. 3G, 4G or similar networks), Wi-Fi, Wi-Max, or any other communication medium that may provide access to a network such as the Internet or a private network. Examples of user devices include mobile phones (e.g. cellular phones), PDAs, tablet computers, net books, laptop computers, personal music players, hand-held specialized readers, etc. Further examples of user devices include wearable devices, such as smart watches, fitness bands, ankle bracelets, rings, earrings, etc., as well as automobiles with remote communication capabilities. A user device may comprise any suitable hardware and software for performing such functions, and may also include multiple devices or components (e.g. when a device has remote access to a network by tethering to another device--i.e. using the other device as a modem--both electronic devices taken together may be considered a single user device).

A credential may be any suitable information that serves as reliable evidence of worth, ownership, identity, or authority. A credential may be a string of numbers, letters, or any other suitable characters that may be present or contained in any object or document that can serve as confirmation.

An application may be computer code or other data stored on a computer readable medium (e.g. memory element or secure element) that may be executable by a processor to complete a task.

A token may define a threshold limit to be applied to a transfer channel. The threshold limit defined by the token may be associated with a participant of the transfer channel. A token may define an amount associated with cybercurrency and/or fiat currency. In some embodiments, a token may be backed by an amount of fiat currency or the token may not be backed with fiat currency. In at least one embodiment, a token may be generated such that the amount associated with the token may become unspendable by a user (e.g., a blockchain user). For example, a blockchain provider may document in a ledger that the user has received a token and may reserve the amount corresponding to the token within the ledger such that the user cannot spend the reserved amount more than once.

A token request message may be an electronic message for requesting a token. A token request message may include information usable for identifying a payment account and/or information for generating a token. For example, a token request message may include payment credentials, user identification information (e.g. a name, alphanumeric identifier, a user device identifier, etc.), an amount associated with a requested token, a transfer channel limit (e.g., a threshold limit associated with the transfer channel and/or a user of the transfer channel), a cryptogram, and/or any other suitable information.

A token response message may be a message that responds to a token request. A token response message may include an indication that a token request was approved or denied. A token response message may also include a token (e.g., an amount and/or threshold limit), user identification information (e.g. a name, alphanumeric identifier, a user device identifier, etc.), a transfer channel limit (e.g., a threshold limit associated with the transfer channel and/or a user of the transfer channel), a cryptogram, and/or any other suitable information.

A transfer channel request message can be an electronic message utilized to request a transfer channel. In some embodiments, a transfer channel request message may include a channel initiator (e.g., a user identifier, a user device identifier, etc.) and any suitable number of channel participants (e.g., one or more other user identifiers, one or more other user device identifiers, etc.). In at least one example, the transfer channel request message may include a data field associated with a threshold limit for the transfer channel and/or a data field associated with a threshold limit associated with one or more users/user devices. In some embodiments, a transfer channel request message may be signed using a private key associated with the user/user device, such that it may be verified using a public key associated with the user/user device, as appropriate. A transfer channel request message may include a request type indicator. The request type indicator may indicate that the request is to open or the request is to close a data transfer channel. In at least one example, a transfer channel request message that indicates that a user is requesting to close a channel may be referred to as a close channel request message. In at least one example, a transfer channel request message that indicates that a user is requesting to open a channel may be referred to as an open channel request message.

A transfer channel response message can be an electronic message utilized to respond to a transfer channel request message. In some embodiments, a transfer channel response message may include a channel initiator (e.g., a user identifier, a user device identifier, etc.) and any suitable number of channel participants (e.g., one or more other user identifiers, one or more other user device identifiers, etc.). In at least one example, the transfer channel response message may include a data field associated with a threshold limit for the transfer channel, a data field associated with a threshold limit associated with one or more users/user devices, a token (e.g., an amount and/or a threshold limit) associated with the transfer channel and/or a user device, and/or a public/private key associated with the user/user device. In at least one embodiment, the transfer channel response message may indicate a response indicator that indicates whether channel creation was successful or unsuccessful. A transfer channel response message may include a request type indicator. The request type indicator may indicate that the response relates to request to open or the response relates to a request to close a data transfer channel. In at least one example, a transfer channel response message that indicates a response to a request to close a channel may be referred to as a close channel response message. In at least one example, a transfer channel response message that indicates a response to a request to open a channel may be referred to as an open channel response message.

As discussed earlier herein, a blockchain can be a distributed database that maintains a continuously-growing list of records secured from tampering and revision. A blockchain may include a number of blocks of interaction records. Each block in the blockchain can contain also include a timestamp and a link to a previous block. For example, each block may include or be appended to a hash of the previous block. Stated differently, interaction records in a blockchain may be stored as a series of blocks, or permanent files that include a record of a number of transactions occurring over a given period of time. Blocks may be appended to a blockchain by an appropriate node after it completes the block and the block is validated. In embodiments of the invention, a blockchain may be distributed, and a copy of the blockchain may be maintained at each node in a verification network. Any node within the verification network may subsequently use the blockchain to verify transactions. The security of a blockchain may be obtained using a cryptographic scheme.

A blockchain provider system can be an electronic device configured to provide blockchain functionality. The blockchain provider system can include a single device or multiple devices configured to maintain aspects of the blockchain (e.g., one or more ledgers, token management, etc.). In some examples, the blockchain provider system may additionally provide token management functionality. Thus, in some embodiments, it is contemplated that blockchain and token management functionality may be commonly performed by a blockchain provider system.

A data transfer request message can be an electronic message utilized to request a data transfer. In some examples, a data transfer request message may be initiated by a user device (e.g., a user device operated by an individual). The data transfer request message may indicate a recipient of the data transfer (e.g., another individual associated with a different user device). For example, another electronic device (e.g., a server, another electronic device operated by another individual/entity) and/or a user (e.g., an individual and/or entity) may be designated as the recipient of the data transfer request message. The data transfer request message may indicate a value associated with the data transfer. By way of example, the value may indicate a monetary amount, a digital asset amount, a number of points (e.g., reward points, a score, etc), or any suitable value/denomination of transferable data. In at least one example, the data transfer request message may include data fields including, but not limited to, an initiator identifier data field, a recipient identifier data field, a transfer value data field, a digital signature data field, a net transaction value, a timestamp data field, and the like. In at least one example, a data transfer request message may be initiated by a data processing computer. In some examples, the net transaction value may be in a format suitable for immediate recordation within a ledger managed by a blockchain provider system. In some embodiments, a data transfer request message may be signed using a private key associated with the user/user device, such that it may be verified using a public key associated with the user/user device, as appropriate.

A data transfer response message can be an electronic message utilized to respond to a data transfer request message. In some examples, a data transfer response message may be initiated by a user device (e.g., an electronic device operated by an individual and/or an entity). The data transfer response message may indicate the initiator of a corresponding data transfer request message. For example, another user device (e.g., a server, another electronic device operated by another individual/entity) and/or a user (e.g., an individual and/or entity) may be designated as the initiator of the data transfer request message corresponding to the data transfer response message. The data transfer response message may indicate a value of the data transfer (e.g., a value received from a corresponding data transfer request message). By way of example, the value may indicate a monetary amount, a digital asset amount, a number of points (e.g., reward points, a score, etc), or any suitable value/denomination of transferable data. In at least one example, the data transfer response message may include data fields including, but not limited to, an initiator identifier data field, a recipient identifier data field, a transfer value data field, a digital signature data field, a timestamp data field, a response code, and the like. In at least one embodiment, the data transfer response message may be digitally signed with a private key of the sender, while in other embodiments a data request response message may not be digitally signed. In some examples, the response code may indicate whether or not the data transfer request has been approved or declined.

An electronic record may be any record of one or more transactions stored electronically. For example, an electronic record may comprise a number of interaction records associated with one or more identities (e.g., two or more users, two or more user devices, etc.). In some embodiments, an electronic record may be utilized to record each of the interaction records received and/or transmitted to/from two or more electronic devices. In some embodiments, an electronic record may be associated with one or more threshold limits. Each threshold limit may indicate a value that is not to be exceeded by data transactions initiated by a particular user and/or a transfer channel. For example, an electronic record associated with two users/electronic devices, may further be associated with two threshold limits. The first threshold limit may indicate a value that is not to be exceeded by data transactions initiated by the first user, while the second threshold limit may indicate a second value that is not to be exceeded by data transactions initiated by the second user. In at least one example, an electronic record may maintain an association between a public key and a user device and/or an association between a token (e.g., an amount and/or threshold limit) maintained by a blockchain provider and a user device.

A cryptographic key may be any string of bits used by a cryptographic algorithm to transform plain text into cipher text or vice versa. Cryptographic keys may include symmetric and asymmetric keys. A cryptographic key may be used to sign data transfer request/response messages. For example, a data transfer request/response message may be signed using a private key. The signed data transfer request/response message may then be verified using a public key that corresponds to the private key.

A private key is a type of cryptographic key that is kept secret by a party. A private key may be used to sign transactions such that they may be verified by another electronic device. For example, one or more data fields may be used to calculate a hash value. A private key may be utilized by a cryptographic algorithm to sign the hash value such that another electronic device may utilize a public key to verify the signature and the verified values may be compared to the unencrypted data transfer request/response message payload to determine validity and integrity of the data transfer request/response message.

A public key may be a type of cryptographic key that is distributed to, or available to, some entity over than a party holding a corresponding private key. A public key may be made available to electronic devices so that signed transactions associated with the public key may be verified by the electronic devices in a similar manner as discussed above.

An authorization request message may be an electronic message that requests authorization for a transaction. In some embodiments, it is sent to a payment processor network and/or an issuer of a payment card to request authorization for a transaction. An authorization request message according to some embodiments may comply with ISO 8583, which is a standard for systems that exchange electronic transaction information associated with a payment made by a user using a payment device or payment account. The authorization request message may include an issuer account identifier that may be associated with a payment device or payment account. An authorization request message may also comprise additional data elements corresponding to identification information including, by way of example only: a service code, a CVV (card verification value), a dCVV (dynamic card verification value), a PAN (primary account number or account number), a user name, an expiration date, etc. An authorization request message may also comprise transaction information, such as any information associated with a current transaction, such as the transaction amount, merchant identifier, merchant location, acquirer bank identification number (BIN), card acceptor ID, information identifying items being purchased, etc., as well as any other information that may be utilized in determining whether to identify and/or authorize a transaction.

An authorization response message may be a message that responds to an authorization request. In some cases, it may be an electronic message reply to an authorization request message generated by an issuing financial institution or a transaction processing computer. The authorization response message may include, by way of example only, one or more of the following status indicators: Approval—transaction was approved; Decline—transaction was not approved; or Contact center—response pending more information, merchant must call the toll-free authorization phone number. The authorization response message may also include an authorization code, which may be a code that a credit card issuing bank returns in response to an authorization request message in an electronic message (either directly or through the transaction processing computer) to the merchant's access device (e.g. POS equipment) that indicates approval of the transaction. The code may serve as proof of authorization. As noted above, in some embodiments, a transaction processing computer may generate or forward the authorization response message to the merchant.

A user may include an individual and/or an entity. In some embodiments, a user may be associated with one or more personal accounts and/or electronic devices. The user may also be referred to as a cardholder, account holder, consumer, merchant, or the like.

A data processing computer may be operated by an entity that can provide data processing services. A data processing computer may provide functionality for processing interaction records and/or managing electronic records associated with one or more users/electronic devices. A data processing computer may be configured to transmit and receive messages (e.g., token request/response messages, data transfer request/response messages, etc.) from two or more electronic devices and/or to/from a blockchain provider.

A server computer may include a powerful computer or cluster of computers. For example, the server computer can be a large mainframe, a minicomputer cluster, or a group of servers functioning as a unit. In one example, the server computer may be a database server coupled to a Web server. The server computer may be coupled to a database and may include any hardware, software, other logic, or combination of the preceding for servicing the requests from one or more client computers. The server computer may comprise one or more computational apparatuses and may use any of a variety of computing structures, arrangements, and compilations for servicing the requests from one or more client computers.

General Implementations

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

The drawings described above and the written description of specific structures and functions below are not presented to limit the scope of what has been invented or the scope of the appended claims. Rather, the drawings and written description are provided to teach any person skilled in the art to make and use the inventions for which patent protection is sought. Those skilled in the art will appreciate that not all features of a commercial implementation of the inventions are described or shown for the sake of clarity and understanding.

Persons of skill in this art will also appreciate that the development of an actual commercial implementation incorporating aspects of the inventions will require numerous implementation-specific decisions to achieve the developer's ultimate goal for the commercial implementation. Such implementation-specific decisions may include, and likely are not limited to, compliance with system-related, business-related, government-related and other constraints, which may vary by specific implementation, location and from time to time. While a developer's efforts might be complex and time-consuming in an absolute sense, such efforts would be, nevertheless, a routine undertaking for those of skill in this art having benefit of this disclosure.

It should be understood that the implementations disclosed and taught herein are susceptible to numerous and various modifications and alternative forms. Thus, the use of a singular term, such as, but not limited to, “a” and the like, is not intended as limiting of the number of items. Also, the use of relational terms, such as, but not limited to, “top,” “bottom,” “left,” “right,” “upper,” “lower,” “down,” “up,” “side,” and the like, are used in the written description for clarity in specific reference to the drawings and are not intended to limit the scope of the invention or the appended claims.

Particular implementations are described with reference to block diagrams and/or operational illustrations of methods. It should be understood that each block of the block diagrams and/or operational illustrations, and combinations of blocks in the block diagrams and/or operational illustrations, may be implemented by analog and/or digital hardware, and/or computer program instructions. Computer programs instructions for use with or by the implementations disclosed herein may be written in an object oriented programming language, conventional procedural programming language, or lower-level code, such as assembly language and/or microcode. The program may be executed entirely on a single processor and/or across multiple processors, as a stand-alone software package or as part of another software package. Such computer program instructions may be provided to a processor of a general-purpose computer, special-purpose computer, ASIC, and/or other programmable data processing system.

The executed instructions may also create structures and functions for implementing the actions specified in the mentioned block diagrams and/or operational illustrations. In some alternate implementations, the functions/actions/structures noted in the drawings may occur out of the order noted in the block diagrams and/or operational illustrations. For example, two operations shown as occurring in succession, in fact, may be executed substantially concurrently or the operations may be executed in the reverse order, depending on the functionality/acts/structure involved.

The term “computer-readable instructions” as used above refers to any instructions that may be performed by a computer processor and/or other components. Similarly, the term “computer-readable medium” refers to any storage medium that may be used to store computer-readable instructions. Such a medium may take many forms, including, but not limited to, non-volatile media, volatile media, and transmission media. Non-volatile media may include, for example, optical or magnetic disks, such as the storage device. Volatile media may include dynamic memory, such as main memory. Transmission media may include coaxial cables, copper wire and fiber optics, including wires of the bus. Transmission media may also take the form of acoustic or light waves, such as those generated during radio frequency (RF) and infrared (IR) data communications. Common forms of computer-readable media may include, for example, a floppy disk, a flexible disk, hard disk, magnetic tape, any other magnetic medium, a CD ROM, DVD, any other optical medium, punch cards, paper tape, any other physical medium with patterns of holes, a RAM, a PROM, an EPROM, a FLASH EPROM, any other memory chip or cartridge, a carrier wave, or any other medium from which a computer can read.

While the disclosed implementations have been described with reference to one or more particular implementations, those skilled in the art will recognize that many changes may be made thereto. Therefore, each of the foregoing implementations and obvious variations thereof is contemplated as falling within the spirit and scope of the disclosed implementations, which are set forth in the claims that follow.

Copyright Notice

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

What is claimed:

1. A method for a contact center to store in a blockchain data related to a consumer, the method comprising:
conducting and concluding a communication with the consumer;
creating a record of the communication;
encrypting the record of the communication with a first key, wherein said record can be decrypted using a second key, the first key and second key operable as an encoder/decoder pair of keys for asymmetric cryptography; and
updating the blockchain with the record of the communication.

2. The method of claim 1, wherein the consumer is a patient and the contact center is associated with a medical services provider, and the updated blockchain is HIPPA compliant.

3. The method of claim 1, wherein the consumer is a patient and the contact center is associated with a medical insurance provider, and the updated blockchain is HIPPA compliant.

4. The method of claim 1, wherein the consumer is a patient and the contact center is associated with a pharmacy, and the updated blockchain is HIPPA compliant.

5. The method of claim 1, wherein the consumer is a medical service provider and the contact center is associated with a medical insurance provider, and the updated blockchain is HIPPA compliant.

6. The method of claim 1, wherein the consumer is a medical service provider and the contact center is associated with a pharmacy, and the updated blockchain is HIPPA compliant.

7. The method of claim 1, wherein the consumer is a medical insurance provider and the contact center is associated with a pharmacy, and the updated blockchain is HIPPA compliant.

8. A system for a contact center to store in a blockchain data related to a consumer, the system comprising:
a memory;
a processor operatively coupled to the memory for:
conducting and concluding a communication with the consumer;
creating a record of the communication;
encrypting the record of the communication with a first key, wherein said record can be decrypted using a second key, the first key and second key operable as an encoder/decoder pair of keys for asymmetric cryptography; and
updating the blockchain with the record of the communication.

9. The system of claim 8, further configured such that wherein the consumer is a patient and the contact center is associated with a medical services provider, and the updated blockchain is HIPPA compliant.

10. The method of claim 8, further configured such that the consumer is a patient and the contact center is associated with a medical insurance provider, and the updated blockchain is HIPPA compliant.

11. The method of claim 8, further configured such that the consumer is a patient and the contact center is associated with a pharmacy, and the updated blockchain is HIPPA compliant.

12. The method of claim 8, further configured such that the consumer is a medical service provider and the contact center is associated with a medical insurance provider, and the updated blockchain is HIPPA compliant.

13. The method of claim 8, further configured such that the consumer is a medical service provider and the contact center is associated with a pharmacy, and the updated blockchain is HIPPA compliant.

14. The method of claim 8, further configured such that the consumer is a medical insurance provider and the contact center is associated with a pharmacy, and the updated blockchain is HIPPA compliant.

15. A computer-readable storage medium comprising computer-readable instructions for a contact center to store in a blockchain data related to a consumer, the computer-readable instructions comprising instructions that cause a processor to:
conduct and conclude a communication with the consumer;
create a record of the communication;
encrypt the record of the communication with a first key, wherein said record can be decrypted using a second key, the first key and second key operable as an encoder/decoder pair of keys for asymmetric cryptography; and
update the blockchain with the record of the communication.

16. The computer-readable medium of claim 15, further comprising instructions whereby the consumer is a patient and the contact center is associated with a medical services provider, and the updated blockchain is HIPPA compliant.

17. The computer-readable medium of claim 15, further comprising instructions whereby the consumer is a patient and the contact center is associated with a medical insurance provider, and the updated blockchain is HIPPA compliant.

18. The computer-readable medium of claim 15, further comprising instructions whereby the consumer is a patient and the contact center is associated with a pharmacy, and the updated blockchain is HIPPA compliant.

19. The computer-readable medium of claim 15, further comprising instructions whereby the consumer is a medical service provider and the contact center is associated with a medical insurance provider, and the updated blockchain is HIPPA compliant.

20. The computer-readable medium of claim 15, further comprising instructions whereby the consumer is a medical service provider and the contact center is associated with a pharmacy, and the updated blockchain is HIPPA compliant.
